(19) United States
(12) Patent Application Publication
(10) Pub. No.: US 2005/0239048 A1
(43) Pub. Date: Oct. 27, 2005

(54) Method and apparatus for enhancing biological photon receptors using plasmon resonance

(76) Inventor: Nabil M. Lawandy, Saunderstown, RI (US)

Correspondence Address: KIRKPATRICK & LOCKHART NICHOLSON GRAHAM LLP;(FORMERLY KIRKPATRICK & LOCKHART LLP), 75 STATE STREET, BOSTON, MA, 02109-1808, US

(21) Appl. No.: 11/102,609

(22) Filed: Apr. 8, 2005

Related U.S. Application Data
(60) Provisional application No. 60/561,068, filed on Apr. 9, 2004.
(60) Provisional application No. 60/561,449, filed on Apr. 12, 2004.
(60) Provisional application No. 60/561,304, filed on Apr. 12, 2004.

(51) Int. Cl. C12Q 1/00; C12M 1/34

(52) U.S. Cl. 435/4; 435/287.2

(57) ABSTRACT

A method for enhancing the light detection capabilities of a chromophore. In one embodiment the method includes the steps of providing a plasmon resonant nanoparticle and placing the nanoparticle in close juxtaposition to the chromophore. In one embodiment the chromophore includes 11-cis-retinal and the nanoparticle is a metal such as gold. In one embodiment the chromophore is chlorophyll. In another aspect the invention relates to an enhanced rhodopsin having a chromophore and a plasmon resonant nanoparticle in close juxtaposition with the chromophore. Yet another aspect of the invention is a method of enhancing vision in an eye including providing a plasmon resonant nanoparticle and placing the nanoparticle in close juxtaposition to a retinal molecule in the eye.

### RELATED APPLICATIONS

This application claims the benefit under 35 U.S.C. § 119(e) to U.S. Provisional Application No. 60/561,068 filed Apr. 9, 2004 entitled PLASMON ENHANCED PHOTORECEPTORS FOR VISION, U.S. Provisional Application No. 60/561,449 filed Apr. 12, 2004 entitled PLASMON ENHANCED PHOTORECEPTORS FOR VISION, and U.S. Provisional Application No. 60/561,304 filed Apr. 12, 2004 and entitled PLASMON ENHANCED PHOTOSYNTHESIS.

### FIELD OF THE INVENTION

The invention relates to the field of biological photon receptors, and more specifically to photon receptor enhancement.

### BACKGROUND OF THE INVENTION

A plasmon is a density wave of charge carriers which form at the interface of a conductor and a dielectric. Plasmons determine, to a degree, the optical properties of conductors, such as metals. Plasmons at a surface can interact strongly with the photons of light, forming a plasmon-polariton. Plasmon excitations at interfaces with dimensions comparable to or significantly smaller than the wavelength of excitation do not propagate and are localized. In ionic materials, surface phonons can produce a negative dielectric response and result in phonon-polaritons. Small scale dimensions lead to localized plasmon and phonon polaritons.

Such localized surface plasmons have been observed since the time of the Romans, who used gold and silver nanoparticles to create colored glass objects such as the Lycurgus Cup (4th Century A.D.). A gold sol in the British museum, created by Michael Faraday in 1857, is still exhibiting its red color due to the plasmon resonance at ˜530 nm.

Localized surface plasmon resonances are associated with giant enhancements of field amplitudes in regions near plasmon resonant particles. For example, gold nanoparticles exhibit the well known Tyndal resonance whereby there is a large absorption in the green region which results in a gold colloid appearing red. The field inside and at the surface of the particle in this case is enhanced by several orders of magnitude and is only limited by the complex dielectric response which remains after the resonance is created when the real parts of the dielectric function approach zero. For any metallic particle in a medium with index of refraction of unity, the plasmon resonance occurs at ωᵣ˜0.58 ωₚ, where ωₚ is the bulk plasmon frequency of the metal.

The field enhancement occurs very near the particle and decays rapidly, typically as 1/R³ for the dipolar limit where R is the distance from the center of the plasmon supporting structure. The field enhancement is also a function of the angular coordinates around the particle. The field enhancement can be realized in aggregates, other shapes such as rods, cubes, triangles, as well as composite core-shell versions of all of those. Changing the shape of the particles or using layered structures of metals and dielectrics may be used to tune the plasmon by means other than material response properties, i.e., changing from gold to silver, etc. Such structures which create localized plasmons can thus act as localized optical field concentrators or antennas for suitable frequencies of light.

The eyes of most animals utilize a combination of rods and cones arranged to create sensitivity to light. Rods are significantly more sensitive, but have no color discrimination, while cones specific to red (L-cones), blue (S-cones) and green (M-cones) provide color response. Cones are less sensitive and therefore primarily function in well lit conditions, while the rods are used for very low light level vision. Each photoreceptor contains many (thousands) of visual pigment molecules.

The active visual pigment in the rods and cones of the eye consists of a protein, opsin, reversibly bound to a chromophore, 11-cis-retinal, a derivative of vitamin A. This visual pigment is termed rhodopsin and in the retina sits within the cell membrane of the eye. Each molecule of rhodopsin consists of seven transmembrane portions surrounding the chromophore (11-cis retinal). The chromophore lies horizontally in the cell membrane and is bound at a lysine residue to the seventh helix in the membrane.

There are many situations in which the eye's sensitivity to light is insufficient. These situations range from decreased eye function as in disease states such as “night blindness” to normal eye function in very low light conditions such as in military operations at night.

There exist other biological photon transducers. Photosynthesis is the process that provides energy for almost all of life. Organisms that carry out photosynthesis are called autotrophs and convert light into energy stored in the form of organic compounds. Photosynthesis occurs in the chloroplasts of plant cells, algae and in the cell membranes of certain bacteria. The basic process uses light energy to convert carbon dioxide and water into three carbon sugars and oxygen. Plants use the organic compounds they make during photosynthesis to carry out their life processes. For example, some of the sugars are used to make components of cell walls, while others are used to form starch. Plants may later break down the starch to produce adenosine triphosphate, ATP, to fuel metabolic processes.

Referring to FIG. 1, the initial step in photosynthesis is the absorption of light in the organelles found in plant cells and algae called chloroplasts. A photosynthetic cell contains anywhere from one to several thousand chloroplasts. Chloroplasts are surrounded by two membranes with the inner one folded into many layers. The inner membrane may fuse along the edges to form thylakoids. Thylakoids are disk shaped structures that contain photosynthetic pigments.

Chlorophylls are the most common and important light absorbing pigments in plants and algae. The two most common types of chlorophylls are chlorophyll a and chlorophyll b. The chlorophylls absorb light from the violet to the red parts of the spectrum. Chlorophyll a absorbs less blue light and more red light than chlorophyll b. Only chlorophyll a is directly involved in the light reactions of photosynthesis. Chlorophyll b assists chlorophyll a in capturing light energy and is called an accessory pigment. The thylakoid membrane also has other colored pigments called carotenoids which are found in carrots, bananas, squash and autumn leaves.

Referring to FIG. 2, photosynthesis is based on a series of electron transport steps which combine oxidative and reducing events. Clusters of pigment molecules (a few hundred) in the thylakoid membranes begin the photosynthesis process when accessory pigment molecules absorb light. In each photosystem, the energy from the photon is transferred to other pigment molecules until it reaches a specific pair of chlorophyll a molecules.

The present invention addresses improving the sensitivity to light of various biological photon receptors.

### SUMMARY OF THE INVENTION

The invention, in one aspect, relates to a method for enhancing the light detection capabilities of a chromophore. The method includes the steps of providing a plasmon resonant nanoparticle and placing the nanoparticle in close juxtaposition to the chromophore. In one embodiment the chromophore includes 11-cis-retinal and the nanoparticle is a metal. In one embodiment the metal is a gold particle. In one embodiment the chromophore is chlorophyll.

In another aspect the invention relates to an enhanced rhodopsin having a chromophore and a plasmon resonant nanoparticle in close juxtaposition with the chromophore. In one embodiment the chromophore is retinal and the nanoparticle is a metal, such as gold. In one embodiment the gold is functionalized.

Another aspect of the invention is a method of enhancing vision in an eye including providing a plasmon resonant nanoparticle and placing the nanoparticle in close juxtaposition to a retinal molecule in the eye.

### BRIEF DESCRIPTION OF THE DRAWINGS

These and other aspects of the invention will be better understood by reference to the specification and drawings in which:

FIG. 1 is a schematic diagram of a chloroplast;

FIG. 2 is a schematic diagram of the absorption of a photon by a chloroplast;

FIG. 3a, b is a graph of the absorption of light by the rods and cones of the eye and the plasmon resonance of a gold particle, respectively;

FIG. 4 shows a molecule of rhodopsin positioned in a cell membrane as enhanced by an embodiment of the invention; and

FIG. 5 is a diagram of thiol linkers to a gold particle.

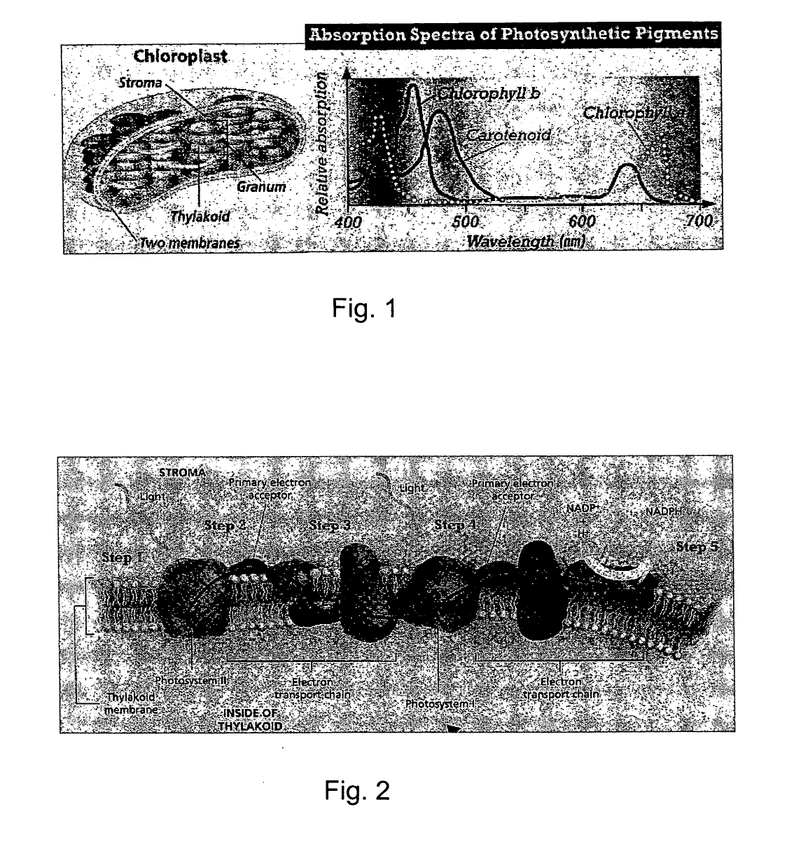

### DESCRIPTION OF A PREFERRED EMBODIMENT

The invention herein relates to the use of local surface plasmon resonances to enhance the local optical fields near the photoreceptors in the eye in order to enhance the sensitivity of the eye. For example, nanoparticles of gold, which has a plasmon resonance in the green region of the spectrum in a water-based environment, may be localized near the receptors in rods or cones to create several orders of magnitude enhancements in the response of those receptors by effectively raising the intensity near the chromophores of retinal

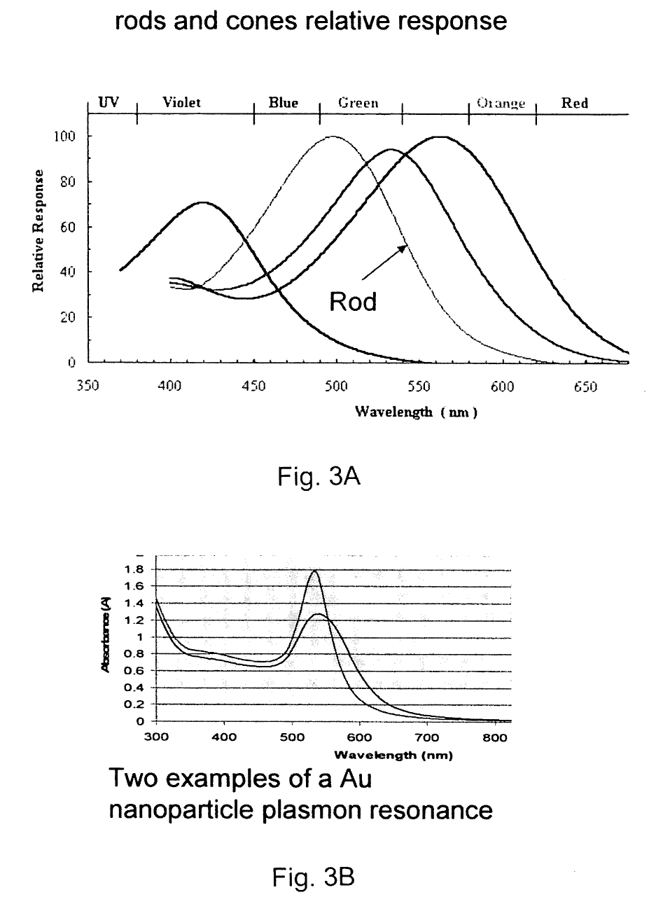

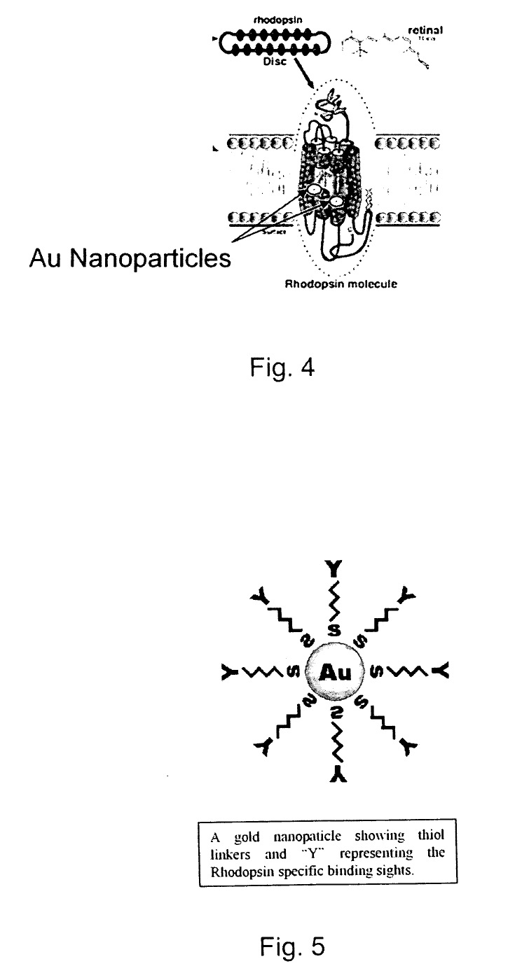

Plasmon enhancement of visual response may be best accomplished by localizing plasmon supporting structures, such as a simple nanoparticle of gold. Referring to FIGS. 3a, b, gold, which is biologically inert, has a plasmon resonance near where the vertebrate vision response peaks, i.e., near the resonance of retinal. Referring also to FIG. 4, this amplification in one embodiment is accomplished using gold particles functionalized to attach at or within tens of nanometers of the lysine residue binding the chromophore to the protein. In one embodiment the gold nanoparticle is functionalized with thiol linkers (FIG. 5). Alternatively, the binding may be directly to the retinal molecules themselves. This technology potentially will allow for nearly blind individuals to see, for normal individuals to see color at very low light levels, and for super-sensitive vision capabilities in very low light conditions such as military applications.

The use of specific plasmon resonances tuned to the S-cones (for example using silver nanoparticles) or the M-cones (using gold nanoparticles) may also allow for the rebalancing of color vision in color blind individuals. Furthermore, some individuals and vertebrate species such as canines have no color vision capability due to the absence of cones. In such situations the use of various plasmon resonances may create wavelength enhanced and specific response in the rods creating a new cognitive version of color vision-capability using only the rod receptors.

One embodiment, the use of a transient effect based on an injection or orally administered nanoparticle drug to create night vision capabilities in soldiers for limited periods of time is envisioned. This may eliminate the need for night vision equipment.

In another embodiment enhanced photoreceptos such rhodopsin molecules may be constructed for technological applications. Such biomimic applications include the creation of high sensitivity photosensors. It should also be noted although the photoreceptor described herein is rhodopsin, other photo-receptive molecules such as bacteiorhodopsin may be used in applications requiring a photon driven proton pump.

These and other aspects and features are further described in more detail below, and additional aspects and features that use the technology described herein will be readily selected by the person of ordinary skill in the art, given the benefit of this disclosure. The embodiments herein described are exemplary and it is intended to limit the invention only by the scope of the claims.

1. A method for enhancing the light detection capabilities of a chromophore comprising the steps of:
providing a plasmon resonant nanoparticle; and
placing the nanoparticle in close juxtaposition to the chromophore.

2. The method of claim 1 wherein the chromophore comprises retinal and the nanoparticle comprises a metal.

3. The method of claim 2 wherein the metal is a gold particle.

4. An enhanced rhodopsin having a chromophore comprising a plasmon resonant nanoparticle in close juxtaposition with the chromophore.

5. The rhodopsin of claim 4 wherein the chromophore is retinal.

6. The rhodopsin of claim 4 wherein the nanoparticle is a metal.

7. The rhodopsin of claim 6 wherein the metal is gold.

8. The rhodopsin of claim 7 wherein the gold is functionalized.

9. The method of claim 1 wherein the chromophore is chlorophyll.

10. A method of enhancing vision comprising:
providing a plasmon resonant nanoparticle; and
placing the nanoparticle in close juxtaposition to a retinal molecule in an eye.
